(19) United States
(12) Patent Application Publication
(10) Pub. No.: US 2007/0129802 A1
(43) Pub. Date: Jun. 7, 2007

(54) Composition and method for producing shapable implants in vivo and implants produced thereby

(75) Inventors: Jagdish M. Jethmalani, San Diego, CA (US); Shiao H. Chang, Pasadena, CA (US); Robert H. Grubbs, Pasadena, CA (US); Julia A. Kornfield, Pasadena, CA (US); Daniel M. Schwartz, San Francisco, CA (US); Christian A. Sandstedt, Pasadena, CA (US); F. R. Christ, Laguna Beach, CA (US)

Correspondence Address: John E. Schneider, Esq.;Fulbright & Jaworski L.L.P., Fulbright Tower, 1301 McKinney, Suite 5100, Houston, TX, 77010-3095, US

(73) Assignee: CALHOUN VISION, INC., Pasadena, CA (US)

(21) Appl. No.: 11/598,168

(22) Filed: Nov. 10, 2006

Related U.S. Application Data
(63) Continuation of application No. 10/103,068, filed on Mar. 21, 2002, now abandoned.
(63) Continuation-in-part of application No. 10/175,552, filed on Jun. 18, 2002.
Application No. 10/175,552 is a continuation-in-part of application No. 10/103,068, filed on Mar. 21, 2002, now abandoned.
Application No. 10/175,552 is a continuation of application No. 09/416,044, filed on Oct. 8, 1999, now Pat. No. 6,450,642.
(60) Provisional application No. 60/277,543, filed on Mar. 21, 2001.
(60) Provisional application No. 60/347,715, filed on Jan. 11, 2002.
(60) Provisional application No. 60/132,871, filed on May 5, 1999.
(60) Provisional application No. 60/115,617, filed on Jan. 12, 1999.

(51) Int. Cl. A61F 2/16; B29D 11/00; A61F 9/013

(52) U.S. Cl. 623/6.22; 623/6.37; 623/6.56; 264/1.7

(57) ABSTRACT

The present invention relates to a method for creating shaped implants, such as intraocular lenses in vivo, as well as the novel implants themselves. Utilizing the method of the invention, it is possible to create an implant in vivo and to adjust either the physical properties such as refractive index, viscosity, etc., mechanical properties such as modulus, tensile strength, tear, etc., or the shape of the implant by noninvasive means. For example, using the method of the patent it is possible to create an intraocular lens in vivo and then adjust the shape and power of the lens through no invasion means. The novel implants are also addressed in this application.

### CROSS-REFERENCE TO RELATED APPLICATIONS

This Application is a Continuation of application Ser. No. 10/103,068 filed on Mar. 21, 2002. Ser. No. 10/103,068 is a Non-Provisional of application 60/277,543 filed on Mar. 21, 2001. Ser. No. 10/103,068 is a Non-Provisional of Provisional (35 USC 119(e)) application 60/347,715 filed on Jan. 11, 2002. This application is also a continuation-in-part of application Ser. No. 10/175,552, which is a continuation of application Ser. No. 09/416,044 filed Oct. 8, 1999, now issued as U.S. Pat. No. 6,450,642 which claims priority from provisional application 60/132,871 filed May 5, 1999 and provisional application 60/115,617 filed Jan. 12, 1999.

### STATEMENT REGARDING FEDERALLY SPONSORED RESEARCH OR DEVELOPMENT

Not applicable.

### REFERENCE TO A COMPACT DISK APPENDIX

Not applicable.

### TECHNICAL FIELD

The present invention relates to a method for creating shaped implants, such as intraocular lenses in vivo, as well as the novel implants themselves. Utilizing the method of the invention, it is possible to create an implant in vivo and to adjust either the physical properties such as refractive index, viscosity, etc., mechanical properties such as modulus, tensile strength, tear, etc., or the shape such as dimensional, radii of curvatures of the implant by noninvasive means. For example, using the method of the patent it is possible to create an intraocular lens in vivo and then adjust the shape and power of the lens through non-invasive means. The novel implants are also addressed in this application.

### BACKGROUND OF THE INVENTION

Approximately two million cataract surgery procedures are performed in the United States annually. The procedure generally involved making an incision in the anterior lens capsule to remove the cataractous crystalline lens and implanting an intraocular lens in its place. The power of the implanted lens is selected (based upon preoperative measurements of ocular length and corneal curvature) to enable the patient to see without additional corrective measures (e.g., glasses or contact lenses). Unfortunately, due to errors in measurement, and/or variable lens positioning and wound healing, about half of all patients undergoing this procedure will not enjoy optimal vision without spectacle correction after surgery. Brandser et al., Acta Ophthalmol. Scand. 75:162-165 (1997); Oshika et al., J. Cataract Refract. Surg. 24:509-514 (1998). Because the power of prior art intraocular lenses generally cannot be adjusted once they have been implanted, the patient typically must choose between replacing the implanted lens with another lens of a different power or be resigned to the use of additional corrective lenses such as glasses or contact lenses. Since the benefits typically do not outweigh the risks of the former, it is almost never done. Another reason for developing a formulation for injecting in the capsular bag of the human eye is for the correction of presbyopia. Presbyopia is the loss of accommodation (inability of a normal eye to form a clear image on the retina) either due to the lens fiber hardening or to the increase in volume with age. Approximately 1.5 billion people worldwide and 89 million people in the United States suffer from presbyopia. Clear lensectomy is gaining popularity where a presbyopic patient opts for replacement of the natural lens with an intraocular lens.

In addition to implanting a prefabricated intraocular lens, several attempts have been made to develop intraocular lenses which can be formed in vivo. For example, U.S. Pat. Nos. 5,411,553 and 5,278,258 disclose an injectable intraocular lens prepared from fast-curing silicone precursor compositions. The patents describe a process whereby a fast-curing silicone composition is injected into the capsular bag. The silicone composition cross-links in vivo to form an intraocular lens.

U.S. Pat. No. 5,391,590 describes an injectable intraocular lens prepared by injecting a mixture of silicone precursors into the capsular bag and cross-linking the precursors in the bag to form a lens. A nonfunctional silicone polymer is added to the mixture to increase the viscosity of the mixture.

U.S. Pat. No. 5,476,515 discloses a method for making an intraocular lens in vivo by injecting collagen-based composition to fill the capsular sac to form a new intraocular lens. The lens is clear and has a refractive index of from 1.2 to 1.6. The collagen is used in its original viscous state or polymerized into a soft gel.

U.S. Pat. No. 5,702,441 discloses a shape transformable intraocular implant which can be readily implanted using an injector. Upon insertion into the capsular bag, the implant regains its original, lens-like shape. U.S. Pat. No. 6,030,416 discloses a similar implant prepared from a stretch-crystallizable, shape-transformable elastomer.

All of these approaches suffer the same disadvantages such as incorrect lens power after curing of the injectable formulation, loss of accommodation, formation of posterior capsular opacification (PCO) over the subsequent period of time as the lenses described above. Once the lens compositions are in place and the lens is formed, there is no way to adjust the shape or refractive index of the lens and thereby adjust the power of the lens.

One solution to this problem can be found in International Patent Application No. PCT/US00/41650 wherein an intraocular lens which is capable of post-fabrication in-vivo power modification is disclosed. The intraocular lens is prepared using a first polymer matrix and a refraction modulating composition that is capable of stimulus-induced polymerization dispersed therein. In one embodiment, when at least a portion of the lens is exposed to an appropriate stimulus, the refraction modulating composition forms a second matrix, causing a thermodynamic inequilibrium which leads to diffusion of the unreacted refraction modulating composition into the exposed region, allowing for an in-vivo precise and accurate modification of the lens power. The base lens is formed in vitro with the adjustment occurring in vivo.

There exists a need however to provide a means for forming an implant, such as an intraocular lens, in vivo, whose refractive power can be modified via change in refractive index and/or shape after the implant is in place. More specifically, a need exists whereby the implant can be modified post-formation without resort to further invasive procedures.

### BRIEF SUMMARY OF THE INVENTION

The present invention relates to a method for creating shapable implants in vivo which involves the injection of polymer precursors capable of forming an implant in vivo in combination with a refraction- and/or shape-modifying composition (“macromer”) into a human body, allowing the polymer precursors to form a polymer matrix with the refraction- and/or shape-modifying composition dispersed therein, exposing at least a portion of the polymer matrix to an external stimulus for a sufficient time to cause the shape and/or refractive index of the polymer matrix to change.

In a preferred embodiment, the implant is an intraocular lens formed by injecting one or more precursors capable of forming a first polymer matrix into the capsular sac in combination with a refraction modulating composition that is capable of stimulus-induced polymerization. The precursors react to form a polymer matrix with the refraction modulating composition dispersed therein. Upon exposure to an external stimulus, the refraction modulating composition cross-links modifying the shape of the polymer matrix and thereby modifying the lens powers of the implant formed. As the injected formulation is a liquid, coating of the capsular bag with this liquid, followed by filling and curing leads to the formation of a tacky material, which adheres to the capsular bag thus may prevent the formation of the posterior capsular opacification (PCO). In addition, the capsular bag remains intact throughout the whole process. Since the bag is attached to the ciliary body via the zonules, this may provide accommodation and correct presbyopia.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a schematic of an implant of the present invention being irradiated in the center followed by irradiation of the entire implant to “lock in” the refractive index modification or the shape of the implant.

FIG. 2 is a pair of photographs showing a human cadaver eye's capsular bag with the cured implant in the capsular bag prior to adjustment.

FIG. 3 is the same cadaver eye's capsular bag with the cured implant after exposure to UV light.

### DETAILED DESCRIPTION OF THE INVENTION

The invention relates to implants which are formed in vivo and which are capable of post-fabrication modification. In one embodiment, it relates to optical elements, such as intraocular lenses which are fabricated in vivo and that are capable of post-fabrication power modifications. More specifically, the present invention relates to intraocular lenses that are formed in vivo and that are capable of being adjusted in situ after formation in the eye. Presbyopia is the loss of accommodation (inability of an normal eye to form a clear image on the retina) either due to the lens fiber hardening or due to the increase in volume with age. Recently, clear or refractive lensectomy where the natural clear lens is replaced with an intraocular lens is gaining popularity for the treatment of presbyopia. However, even after the lens replacement, implantation with an incorrect lens power, wound healing or lens positioning lead to refractive errors. Another approach is to replace the natural lens with an injectable formulation and in-vivo curing of the formulation in the capsular bag. As the natural lens possesses the elastic modulus in the range of 10³-10⁴ Pa and is able to accommodate, formulations could be developed to possess similar elastic modulus. (Murthy, K. S. and Ravi, N. Current Eye Research, 2001, vol. 22(5), pp. 384-393.) Since the capsular bag, the zonules, ciliary muscles and ciliary body remain intact after curing, accommodation may be restored. Several examples of such can be found by work conducted by Nishi and co-workers. In these cases, the capsular bag was filled to various capacities and after curing, it was found that partial accommodation was restored. However, the partial filling of the bag lead to refractive errors mainly hyperopia and posterior capsular opacification (PCO). A possible solution to these problems involves injecting a formulation into the capsular bag. Upon curing it forms a tacky, low modulus matrix that adheres to the bag preventing PCO, providing accommodation as and when the ciliary body and muscles exert force on it. To correct the refractive errors after curing, the material can be adjusted in-vivo by stimulus-induced polymerization.

The implants of the invention comprise a first polymer matrix and a refraction- and/or shape-modifying composition dispersed therein. The first polymer matrix forms the implant framework and is generally responsible for many of its material properties. It comprises at least one component which is capable of forming a polymer matrix at temperatures and conditions encountered in a living organism. The refraction- and/or shape-modifying composition may be a single compound or a combination of compounds that is capable of stimulus-induced polymerization, preferably photopolymerization. As used herein, the term “polymerization” refers to a reaction wherein at least one of the components of the refraction- and/or shape-modifying composition reacts to form at least one covalent or physical bond with either a like component or with a different component. The identities of the first polymer matrix and the refraction- and/or shape-modifying composition will depend on the end use of the implant. However, as a general rule, the first polymer matrix and the refraction- and/or shape-modifying composition are selected such that the compositions are capable of diffusion through the first polymer matrix. Put another way, a loose first polymer matrix will tend to be paired with larger refraction- and/or shape-modifying composition components and a tight first polymer matrix will tend to be paired with smaller refraction- and/or shape-modifying composition components.

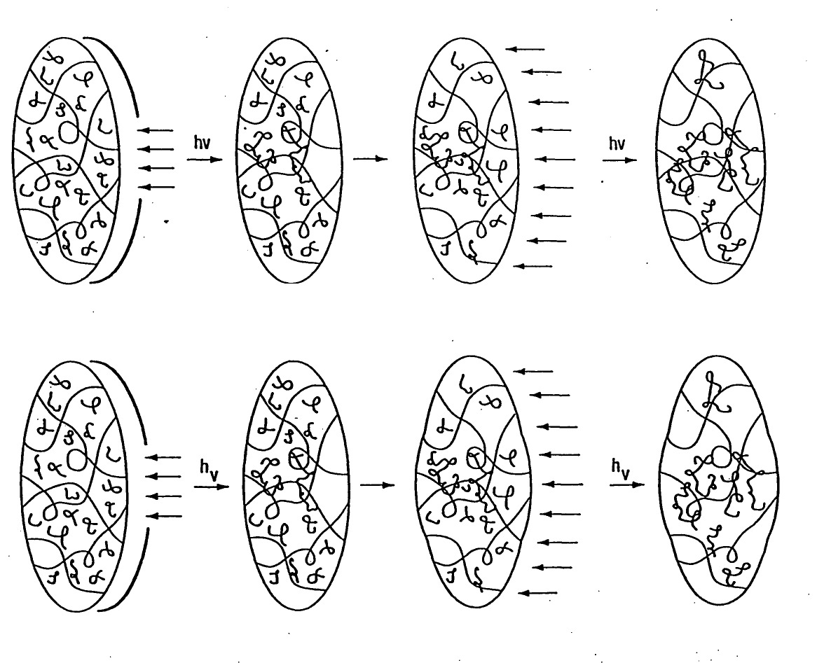

Upon exposure to an appropriate energy source (e.g., heat or light), the refraction- and/or shape-modifying composition typically forms a second polymer matrix in the exposed region on the implant. The presence of the second polymer matrix changes the material characteristics of this portion of the implant. For example, for an intraocular lens, the presence of the second polymer matrix will modulate the refraction capabilities of the lens. In general, the formation of the second polymer matrix in an intraocular lens typically increases the refractive index of the affected portion of the optical element. After exposure, the refraction- and/or shape-modifying composition in the unexposed region will migrate into the exposed region over time. The migration of the shape-modifying composition into the exposed region is time dependent and may be precisely controlled. If enough time is permitted, the refraction- and/or shape-modifying composition components will re-equilibrate and redistribute throughout the implant (i.e., the first polymer matrix, including the exposed region). When the region is re-exposed to the energy source, the shape-modifying composition that has since migrated into the exposed region (which may be less than if the refraction- and/or shape-modifying component composition were allowed to re-equilibrate) polymerizes to further increase the formation of the second polymer matrix. This process (exposure followed by an appropriate time interval to allow for diffusion) may be repeated until the exposed region has reached the desired shape. For an intraocular lens this means that the process is repeated until the optical element has reached the desired property (e.g., power, refractive index, or shape). At any point of this adjustment, the implant is exposed to the energy source to “lock-in” the implant property by polymerizing the remaining refraction- and/or shape-modifying components that are outside the exposed region before the components can migrate into the exposed region. In other words, because freely diffusable refraction- and/or shape-modifying composition components are no longer available, subsequent exposure of the implant to an energy source cannot further change its shape. FIG. 1 illustrates one inventive embodiment—refractive index modulation followed by change in radius of curvature due to swelling (thus lens power modulation) and a lock-in.

The first polymer matrix is a covalently or physically linked structure that functions as an optical element and is formed from a first polymer matrix composition. In general, the first polymer matrix composition comprises one or more monomers that upon polymerization will form the first polymer matrix. The first polymer matrix composition optionally may include any number of auxiliaries that modulate the polymerization reaction or modify one or more properties of the implant. Illustrative examples of suitable first polymer matrix composition monomers include acrylates, methacrylates, phosphazenes, siloxanes, vinyls, urethanes, homopolymers and copolymers thereof. As used herein, a “monomer” refers to any unit (which may itself either be a homopolymer or a copolymer) which may be linked together to form a polymer containing repeating units of the same. If the first polymer matrix composition monomer is a copolymer, it may be comprised of the same or different types of monomers.

In one embodiment, the one or more monomers that form the first polymer matrix are polymerized in vivo and cross-linked in the presence of the refraction- and/or shape-modifying composition. In another embodiment, polymeric starting material that forms the first polymer matrix is cross-linked in the presence of the refraction- and/or shape-modifying composition. Under either scenario, the refraction- and/or shape-modifying composition components must be compatible with and not appreciably interfere with the formation of the first polymer matrix. Similarly, the formation of the second polymer matrix should also be compatible with the existing first polymer matrix. Put another way, the first polymer matrix and the second polymer matrix should not phase separate and the properties of the implant, e.g., light transmission, should not be affected.

As described previously, the refraction- and/or shape-modifying composition may be a single component or multiple components so long as: (i) it is compatible with the formation of the first polymer matrix; (ii) it remains capable of stimulus-induced polymerization after the formation of the first polymer matrix; and (iii) is freely diffusable within the first polymer matrix. In the preferred embodiment, the stimulus-induced polymerization is photo-induced polymerization and the power corrections include hyperopic, myopic, astigmatic, coma, 3^{rd} order spherical and other higher order aberrations.

The inventive implants have numerous applications in the biomedical field. One specific application for the present invention is in vivo-formed medical lenses, particularly as intraocular lenses with the capabilities of accommodation as the presence of the capsular bag exists which are held by ligatures (i.e., the zonules which connect to the capsular bag in the equatorial regions and insert at the other end into the ciliary muscle). Other biomedical application could be in the area of cosmetic implants in human body.

In general, there are two types of intraocular lenses (“IOLs”). The first type of intraocular lens replaces the eye's natural lens. The most common reason for such a procedure is cataracts. The second type of intraocular lens supplements the existing lens and functions as a permanent corrective lens. This type of lens (sometimes referred to as a phakic intraocular lens) is implanted in the anterior or posterior chamber to correct any refractive errors of the eye. In theory, the power for either type of intraocular lens required for emmetropia (i.e., perfect focus on the retina from light at infinity) can be precisely calculated. However, in practice, due to errors in measurement of corneal curvature, and/or variable lens positioning and wound healing, it is estimated that only about half of all patients undergoing IOL implantation will enjoy the best possible vision without the need for additional correction after surgery. Because prior art IOLs are generally incapable of post-surgical power modification, the remaining patients must resort to other types of vision correction such as external lenses (e.g., glasses or contact lenses) or cornea surgery. The need for these types of additional corrective measures is obviated with the use of the intraocular lenses of the present invention. The inventive intraocular lens comprises a first polymer matrix and a refraction modulating composition dispersed therein. The first polymer matrix and the refraction modulating composition are as described above with the additional requirement that the resulting lens be biocompatible.

Illustrative examples of a suitable first polymer matrix include: poly-acrylates such as poly-alkyl acrylates and poly-hydroxyalkyl acrylates; poly-methacrylates such as poly-methyl methacrylate (“PMMA”), poly-hydroxyethyl methacrylate (“PHEMA”), and poly-hydroxypropyl methacrylate (“HPMA”); poly-vinyls such as poly-styrene and poly-N-vinylpyrrolidone (“PNVP”); poly-siloxanes such as poly-dimethylsiloxane, dimethylsiloxane diphenylsiloxane copolymers, dimethylsiloxane methylphenylsiloxane copolymers; poly-phosphazenes; urethanes and copolymers thereof. U.S. Pat. No. 4,260,725 and patents and references cited therein (which are all incorporated herein by reference) provide more specific examples of suitable polymers that may be used to form the first polymer matrix.

In preferred embodiments, the first polymer matrix generally possesses a relatively low glass transition temperature (“T_{g}”) such that the resulting IOL tends to exhibit fluid-like and/or elastomeric behavior, and is typically formed by crosslinking one or more polymeric starting material wherein each polymeric starting material includes at least one crosslinkable group. Illustrative examples of suitable crosslinkable groups include but are not limited to hydride, vinyl, acetoxy, alkoxy, amino, anhydride, aryloxy, carboxy, enoxy, epoxy, halide, isocyano, olefinic, and oxime. In more preferred embodiments, each polymeric starting material includes terminal monomers (also referred to as endcaps) that are either the same or different from the one or more monomers that comprise the polymeric starting material but include at least one crosslinkable group. Consequently, other embodiments include crosslinkers that have reactive groups attached as side-groups along the backbone and/or terminal endcaps. In other words, the terminal monomers begin and end the polymeric starting material and include at least one crosslinkable group as part of its structure. Although it is not necessary for the practice of the present invention, the mechanism for crosslinking the polymeric starting material preferably is different than the mechanism for the stimulus-induced polymerization of the components that comprise the refraction modulating composition. For example, if the refraction modulating composition is polymerized by photo-induced polymerization, then it is preferred that the polymeric starting materials have crosslinkable groups that are polymerized by any mechanism other than photo-induced polymerization.

An especially preferred class of polymeric starting materials for the formation of the first polymer matrix is poly-siloxanes (also know as “silicones”) endcapped with a terminal monomer which includes a crosslinkable group selected from the group comprising acetoxy, amino, alkoxy, halide, hydroxy, vinyl, hydride and mercapto. Because silicone IOLs tend to be flexible and foldable, generally smaller incisions may be used during the IOL implantation procedure. An example of an especially preferred polymeric starting material is bis(diacetoxymethylsilyl)-polydimethylsiloxane (which is poly-dimethylsiloxane that is endcapped with a diacetoxymethylsilyl terminal monomer). Another example involves hydrosilylation reaction between the vinyl- and the hydride-functionalized silicones in presence of a catalyst, preferably a platinum complex and is similar to the compositions described in the U.S. Pat. No. 5,411,553 and others.

In the present invention, the first polymer matrix is formed in vivo. This is accomplished in injecting the precursors for the first polymer matrix as well as the refraction- and/or shape-modifying composition into a body cavity and allowing the precursors of the first polymer matrix to cure in the presence of the refraction- and/or shape-modifying composition. The curing is accomplished through catalytic polymerization of the first and second precursor.

Where the first polymer matrix is a silicone-based matrix, two types of precursors are required to form the first polymer matrix useful in the practice of the invention. The first precursor comprises one or more vinyl-containing polyorganosiloxanes and the second precursors comprise one or more organosilicon compounds having silicon-bonded hydride groups which react with the vinyl groups of the first precursor.

The first precursor preferably has an average of at least two silicone-bonded vinyl radicals per molecule. The number of vinyl radicals can vary from two per molecule. For example the first precursor can be a blend of two or more polyorganosiloxanes in which some of the molecules have more than two vinyl radicals per molecule and some have less than two vinyl radicals per molecule. Although it is not required that the silicon-bonded vinyl radicals be located in the alpha, omega (or terminal) positions, it is preferred that at least some of the vinyl radicals be located at these positions. The vinyl radicals are located at the polymer ends because such polyorganosiloxanes are economical to produce and provide satisfactory products. However, because of the polymeric nature of the first precursor, its preparation may result in products that have some variation in structure, and some vinyls may not be in the terminal position, even if the intent is to have them in these positions. Thus, the resulting polyorganosiloxanes may have a portion of the vinyl radicals located at branch sites.

The polyorganosiloxanes of the first precursor are preferably essentially linear polymers that may have some branching. The polyorganosiloxanes may have silicon-oxygen-silicon backbones with an average of greater than two organo groups per silicon atom. Preferably, the first precursor is made up of diorganosiloxane units with triorganosiloxane units for endgroups, but small amounts of monoorganosiloxane units and SiO₂ may also be present. The organo radicals preferably have less than about 10 carbon atoms per radical and are each independently selected from monovalent hydrocarbon radicals such as methyl, ethyl, vinyl propyl, hexyl and phenyl and monovalent substituted hydrocarbon radicals such as perfluoroalkylethyl radicals. Examples of first precursors include dimethylvinylsiloxy endblocked polydimethylsiloxane, methylphenylvinylsiloxy endblocked polydimethylsiloxane, dimethylvinylsiloxy endblocked polymethyl-(3,3,3-triflouropropyl)siloxane, dimethylsiloxy endblocked polydiorganosiloxane copolymers of dimethylsiloxane units and methylphenylsiloxane units and methylphenylvinylsiloxy endblocked polydiorganosiloxane copolymers of dimethylsiloxane units and diphenylsiloxane units and the like. The polydiorganosiloxane can have siloxane units such as dimethylsiloxane units, methylphenylsiloxane units, methyl-(3,3,3-trifluoropropyl)siloxane units, monomethylsiloxane units, monophenylsiloxane units, dimethylvinylsiloxane units, trimethylsiloxane units, and SiO₂ units. Polyorganosiloxanes of the first precursor can be single polymers or mixtures of polymers. These polymers may have at least fifty percent of the organic radicals as methyl radicals. Many polyorganosiloxanes useful as the first precursor are known in the art and are commercially available. A preferred first precursor is polydimethylsiloxane endblocked with dimethylvinylsiloxy units or methylphenylsiloxy units having a viscosity of from about 500 to 100,000 centipoise at 25° C.

The second precursor includes organosilicon compounds containing at least 2, and preferably at least 3, silicon-bonded hydride groups, i.e., hydrogen atoms, per molecule. Each of the silicon-bonded hydride groups is preferably bonded to a different silicon atom. The remaining valences of the silicon atom are satisfied by divalent oxygen atoms or by monovalent radicals, such as alkyl having from 1 to about 6 carbon atoms per radical, for example methyl, ethyl, propyl, isopropyl, butyl, tertiary butyl, pentyl hexyl, cyclohexyl, substituted alkyl radicals, aryl radicals, substituted aryl radicals and the like. The silicon-bonded hydride group containing organosilicon compounds can be homopolymers, copolymers and mixtures thereof which contain siloxane units of the following types: RSiO_{1.5}, R₂SiO, RHSiO, HsiO_{1.5}, R₂HsiO_{0.5}, H₂SiO RH₂ SiO^{0.5}, and SiO where R is the monovalent radical, for example, as defined above. Examples include polymethylhydrogensiloxane cyclics, copolymers of trimethylsiloxy and methylhydrogensiloxane, copolymers of dimethylsiloxy and methylhydrogensiloxane, copolymers of trimethylsiloxy, dimethylsiloxane and methylhydrogensiloxane, copolymers of dimethylhydrogensiloxane, dimethylsiloxane and methylhydrogensiloxane and the like. Also needed is a crosslinker resin. This resin is a multifunctional vinyl silicone of certain mole. wt., branched structure and functionality. The other crosslinker is the multifunctional silicone hydride of certain mole. wt., branched structure and functionality.

The platinum group metal catalyst component can be any of the compatible platinum group metal-containing catalysis known to catalyze the addition of silicone-bonded hydrogen atoms (hydride groups) to silicon-bonded vinyl radicals. Platinum group metal-containing catalysts can be any of the known forms which are compatible, such as platinic chloride, salts of platinum, chloroplatinic acid and various complexes. The platinum group metal-containing catalyst can be used in any catalytic quantity, such as in an amount sufficient to provide at least about 0.1 ppm weight of platinum group metal (calculated as elemental metal) based on the combined weight of the first and second precursors. Preferably, at least about 10 ppm, for example, at least about 20 ppm or at least 30 ppm or at least about 40 ppm, by weight of platinum group metal based on the combined weight of the first and second precursors is used.

The refraction- and/or shape-modifying composition that is used in fabricating implants of the invention is as described above except that it has the preferred requirement of biocompatibility. The refraction- and/or shape-modifying composition is capable of stimulus-induced polymerization and may be a single component or multiple components so long as: (i) it is compatible with the formation of the first polymer matrix; (ii) it remains capable of stimulus-induced polymerization after the formation of the first polymer matrix; (iii) it is freely diffusable within the first polymer matrix. In general, the same type of monomer that is used to form the first polymer matrix may be used as a component of the shape-modifying composition. The monomers will often contain functional groups that are capable of stimulus-induced polymerization. However, because of the requirement that the refraction- and/or shape-modifying composition monomers must be diffusable within the first polymer matrix, the refraction- and/or shape-modifying composition monomers generally tend to be smaller (i.e., have lower molecular weights) than the first polymer matrix network, i.e., the diffusible materials have to be of mw less than for instance the mw between crosslinks of the first polymer matrix. In addition to the one or more monomers, the refraction- and/or shape-modifying composition may include other components such as initiators and sensitizers that facilitate the formation of the second polymer matrix. In addition, to provide the UV-blocking properties similar to the natural eye, UV-absorbers may also be incorporated as a component of the refraction- and/or shape-modifying composition.

In preferred embodiments, the stimulus-induced polymerization is photopolymerization. In other words, for the one or more monomers that comprise the refraction- and/or shape modulating composition, each preferably includes at least one functional group that is capable of photopolymerization. Illustrative examples of such photopolymerizable groups include but are not limited to acrylate, allyloxy, cinnamoyl, methacrylate, stibenyl, and vinyl. In more preferred embodiments, the refraction- and/or shape-modifying composition includes a photoinitiator (any compound used to generate free radicals) either alone or in the presence of a sensitizer and UV-absorbers. Examples of suitable photoinitiators include acetophenones (e.g., substituted haloacetophenone, and diethoxyacetophenone); 2,4-dichloromethyl-1,3,5-triazines; benzoin methyl ether; and o-benzoly oximino ketone and silicone derivatives thereof. Examples of suitable sensitizers include p-(dialkylamino)aryl aldehyde; N-alkylindolylidene; and bis[p-(dialkylamino)benzylidiene]ketone and silicone derivatives thereof. Examples of UV-absorbers include but are not limited to the benzophenones and their derivatives, benzotriazoles and their derivatives, and others that are known in the art of UV-blocking materials.

As noted above, the implants of the invention are often used as IOLs. Because of the preference for flexible and foldable IOLs, an especially preferred class of refraction- and/or shape-modifying composition monomers is poly-siloxanes endcapped with a terminal siloxane moiety that includes a photopolymerizable group. An illustrative representation of such a monomer is:
X—Y—X¹
wherein Y is a siloxane which may be a monomer, a homopolymer or a copolymer formed from any number of siloxane units, and X and X1 may be the same or different and are each independently a terminal siloxane moiety that includes a photopolymerizable group. An illustrative example of Y includes: 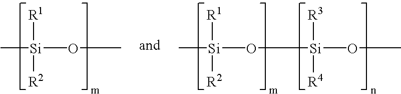
wherein: m and n are independently each an integer and R1, R2, R3, and R4 are independently each hydrogen, alkyl (primary, secondary, tertiary, cyclo), aryl, or heteroaryl. In preferred embodiments, R¹, R², R³, and R⁴ is a C₁-C₁₀ alkyl or phenyl. Because shape-modifying composition monomers with a relatively high aryl content have been found to produce larger changes in the refractive index of the inventive lens, it is generally preferred that at least one of R¹, R², R³, and R⁴ is an aryl, particularly phenyl. In more preferred embodiments, R¹, R², and R³ are the same and are methyl, ethyl or proply and R⁴ is phenyl.

Illustrative examples of X and X¹ (or X¹ and X depending on how the RSMC polymer is depicted) are 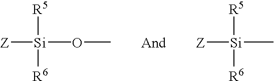
respectively wherein:

R⁵ and R⁶ are independently each hydrogen, alkyl, aryl, or heteroaryl; and Z is a photopolymerizable group.

In preferred embodiments, R⁵ and R⁶ are independently each a C₁-C₁₀ alkyl or phenyl and Z is a photopolymerizable group that includes a moiety selected from the group consisting of acrylate, allyloxy, cinnamoyl, methacrylate, stibenyl, and vinyl. In more preferred embodiments, R⁵ and R⁶ is methyl, ethyl, or propyl and Z is a photopolymerizable group that includes an acrylate or methacrylate moiety.

In especially preferred embodiments, the refraction- and/or shape-modifying composition monomer is of the following formula: 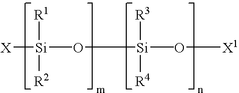
wherein X and X¹ are the same and R¹, R², R³, and R⁴ are as defined previously. Illustrative examples of such shape-modifying composition monomers include dimethylsiloxane-diphenylsiloxane copolymer endcapped with a vinyl dimethylsilane group; dimethylsiloxane-methylphenylsiloxane copolymer endcapped with a methacryloxypropyl dimethylsilane group; and dimethylsiloxane endcapped with a methacryloxypropyldimethylsilane group. Although any suitable method may be used, a ring-opening reaction of one or more cyclic siloxanes in the presence of triflic acid has been found to be a particularly efficient method of making one class of inventive shape-modifying composition monomers. Briefly, the method comprises contacting a cyclic siloxane with a compound of the formula: 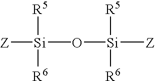
in the presence of triflic acid wherein R5, R6, and Z are as defined previously. The cyclic siloxane may be a cyclic siloxane monomer, homopolymer, or copolymer. Alternatively, more than one cyclic siloxane may be used. For example, a cyclic dimethylsiloxane tetramer and a cyclic methyl-phenylsiloxane trimer/tetramer are contacted with bis-methacryloxypropyltetramethyldisiloxane in the presence of triflic acid to form a dimethyl-siloxane methyl-phenylsiloxane copolymer that is endcapped with a methacryloxypropyl-dimethylsilane group, an especially preferred shape-modifying composition monomer.

In practice, a body cavity is prepared for formation of the implant. In the case of an IOL, this is often accomplished by first removing the existing lens by phaco-emulsification leaving the lens capsule intact except for the flap necessary to insert the phaco tip. The monomers or polymer precursors necessary to form the first polymer matrix as well as the refraction or shape-modifying composition are mixed and precured and are injected into the body cavity such that the first polymer matrix is formed in the body cavity. Alternately, the first polymer precursor and the refraction- and/or shape modifying composition are mixed, degassed, transferred to syringe, and cooled to a temperature (between −10° to 0° C.) at which the first polymer matrix crosslinking is inhibited. The shape-modifying composition monomers as well as any initiators required to form the second polymer matrix and other components, such as UV absorber, are mixed with the first polymer matrix monomers before injection into the body cavity.

For the implants of the invention, the curing temperature for the first polymer matrix is the physiological temperature of the eye, for example, in humans in the range of about 35° C. to about 37° C. Lack of mobility of the injected composition preferably occurs about 20 minutes after injection, more preferably within about 10 minutes. Final cure preferably occurs within about 6 hours, more preferably within about 2 hours of injection.

In one embodiment of the invention, the first and second precursors are separated into two discrete compositions. The first composite comprises the first precursor combined with the refraction- and/or shape-modifying composition (macromer), photoinitiator and, where desired, an UV-absorber are combined. In the second composite, the second precursor and catalyst are combined. Alternatively, the catalyst can be combined with the first precursor and the other components combined with the second precursor. The key is to keep the first and second precursors and the catalyst separate until just before the materials are injected into the body cavity.

Implantation of the injectable implants of the invention is relatively straightforward. For example, in the case of an intraocular lens, the lens is first removed by phaecoemulsification. The components of the intraocular lens are then injected into the capsular bag using a syringe. The capsular bag is then sealed and the lens is allowed to cure. A preferred means of sealing the capsular bag is through the use of a plug as described by Nishi et al., in J. Cataract Surg., 1998, 24, 975-982 and in Arch. Ophthalmol., 116, 1358-1361. Alternatively, the lens can be injected into an endocapsular balloon that is placed in the capsular bag. This procedure is similar to that described by Nishi et al. in J. Cataract Surg., 1997, 23, 1548-1555.

A preferred way to prepare the implants of the present invention is through use of a multichamber syringe which keeps the individual components separate until just before the components are injected into the body cavity. While each component may be injected separately, some components may be combined provided that they do not interact such that they fail to perform as required once they are injected into the body cavity. For example, where the first polymer matrix is formed from two separate monomers in the presence of a catalyst, one chamber of the syringe will contain the first monomer and the second chamber will contain the other monomer. The catalyst can be combined with either monomer unless the catalyst will cause the monomer to polymerize in the chamber. Additional components can be combined in one of the other chambers. For example, the refraction- and/or shape-modifying components can be placed in either chamber as well as any other additives. In the case of intraocular lenses, the additives can include UV absorber such as benzotriazoles, benzophenones, phenylesters, cinnamic acid and derivatives and nickel-containing compounds.

A key advantage of the implants of the present invention is that an implant property may be modified after implantation within the body. For example, in the case of an IOL, any errors in the power calculation due to imperfect corneal measurements and/or variable lens positioning and wound healing may be modified in a post-surgical outpatient procedure. Typical lenses produced in this manner have a refractive index of from about 1.40-1.50.

In addition to the change in the IOL refractive index, the stimulus-induced formation of the second polymer matrix has been found to affect the IOL power by altering the lens curvature in a predictable manner. As a result, both mechanisms may be exploited to modulate an IOL property, such as power, after it has been implanted within the eye. In general, the method for implementing an inventive implant having a first polymer matrix and a shape-modifying composition dispersed therein, comprises:

(a) exposing at least a portion of the implant to an external stimulus whereby the stimulus induces the polymerization of the shape-modifying composition. If after formation of the implant and wound healing, no implant property needs to be modified, then the exposed portion is the entire implant. The exposure of the entire implant will lock in the then-existing properties of the implanted implant. However, if an implant characteristic such as the power of an IOL needs to be modified, then only a portion of the implant (something less than the entire implant) would be exposed. In one embodiment, the method of implementing the inventive implant further comprises:

(b) waiting an interval of time; and

(c) re-exposing the portion of the implant to the stimulus.

This procedure generally will induce the further polymerization of the refraction modulating composition within the exposed implant portion. Steps (b) and (c) may be repeated any number of times until the implant has reached the desired implant characteristic. At this point, the method may further include the step of exposing the entire implant to the stimulus to lock-in the desired lens property.

In another embodiment wherein a lens property needs to be modified, a method for implementing an inventive IOL comprises:

(a) exposing a first portion of the lens to a stimulus whereby the stimulus induces the polymerization of the refraction modulating composition; and

(b) exposing a second portion of the lens to the stimulus.

The first lens portion and the second lens portion represent different regions of the lens although they may overlap. Optionally, the method may include an interval of time between the exposures of the first lens portion and the second lens portion. In addition, the method may further comprise re-exposing the first lens portion and/or the second lens portion any number of times (with or without an interval of time between exposures) or may further comprise exposing additional portions of the lens (e.g., a third lens portion, a fourth lens portion, etc.). Once the desired property has been reached, then the method may further include the step of exposing the entire lens to the stimulus to lock-in the desired lens property.

In general, the location of the one or more exposed portions will vary depending on the type of refractive error being corrected. For example, in one embodiment, the exposed portion of the IOL is the optical zone which is the center region of the lens (e.g., between about 4 mm and about 5 mm in diameter). Alternatively, the one or more exposed lens portions may be along the IOL's outer rim or along a particular meridian. In preferred embodiments, the stimulus is light. In more preferred embodiments, the light is from a laser or lamp source. The intensity profile could be of any shape or size to correct myopia, hyperopia, astigmatism and other higher order aberrations. The intensity profile may be generated by directing the light source through a spatial light modulator (SLM), liquid crystal displays (LCD), deformable mirrors similar to those used in adaptive optics, digital light processor (DLP), digital micro-mirror device (DMD), etc. and those known in the art of display technologies.

In summary, the present invention relates to a novel implant that comprises (i) a first polymer matrix and (ii) a refraction- and/or shape-modifying composition that is capable of stimulus-induced polymerization dispersed therein. When at least a portion of the implant is exposed to an appropriate stimulus, the refraction- and/or shape-modifying composition forms a second polymer matrix. The amount and location of the second polymer matrix modifies a property of the implant such as the power of an optical element by changing its refractive index and/or by altering its shape.

### EXAMPLE I

By mixing 0.23% of initiator (Irgacure 651) in 30% 1,000 g/mole bismethacrylate endcapped polydimethylsiloxane (macromer), the refraction modulating composition was made. To this mixture, 70% of 36,000 g/mole diacetoxymethylsilyl endcapped polydimethylsiloxane (matrix) was added and the entire composition mixed well. The mixture was degassed under pressure in a vacuum oven for 10 minutes and then transferred to a syringe. Using 20-gauge needle, the final formulation was injected in a bubble of a bubble wrap plastic and allowed to cure at room temperature for a period of 24 hours. The cured material conformed to the shape of the bubble and possessed desired mechanical properties.

### EXAMPLE II

By mixing 0.23% of initiator (Irgacure 651), 0.02% of UV-absorber 2(2′-hydroxy-3′-t-butyl-5′-vinylphenyl)-5-chloro-2h-benzotriazole, (UVAM) in 30% 1000 g/mole bismethacrylate endcapped dimethylsiloxane methylphenylsiloxane copolymer (macromer), the refraction modulating composition was made. To this mixture, 35% of Part B of commercial silicone (MED-6820, NuSil) and 1-5% of crosslinker methylhydrocyclosiloxane was added and the entire composition mixed and degassed under pressure in a vacuum oven for 10 minutes. Finally to this mixture, 35% of Part A of commercial silicone (MED-6820, NuSil) and a drop of Platinum catalyst (PC075, United Chemical Technology) were added and the composition mixed and degassed under pressure. The final formulation was transferred to a syringe. Using 20 Gauge needle, it was injected in a bubble of a bubble wrap plastic or in an oral dosage capsule that was drained of its pharmaceutical contents Vitamin E. The silicone was allowed to cure for a period of 24 hours at 40° C. The cured material conformed to the shape of the capsule and possessed desired mechanical properties.

### EXAMPLE III

By mixing 0.23% of initiator (Irgacure 651), 0.02% of UV-absorber (UVAM) in 30% 1000 g/mole bismethacrylate endcapped polydimethylsiloxane (macromer), the refraction modulating composition was made. To this mixture, 35% of Part B of commercial silicone (MED-6033, NuSil) and 1-5% of crosslinker methylhydrocyclosiloxane was added and the entire composition mixed and degassed under pressure in a vacuum oven for 10 minutes. Finally to this mixture, 35% of Part A of commercial silicone (MED-6033, NuSil) and a drop of Platinum catalyst (PC075, United Chemical Technology) were added and the composition mixed and degassed under pressure. The final formulation was transferred to a syringe. Using 20-gauge needle, it was injected in a bubble of a bubble wrap plastic or in an oral dosage capsule that was drained of its pharmaceutical content Vitamin E. The silicone was allowed to cure for a period of 24 hours at 35° C. The cured material conformed to the shape of the capsule and possessed desired mechanical properties.

### EXAMPLE IV

By mixing 0.23% of initiator (Irgacure 651), 0.02% of UV-absorber (UVAM) in 30% 1000 g/mole bismethacrylate endcapped polydimethylsiloxane (macromer), the refraction modulating composition was made. To this mixture, 35% of base polymer (similar to that used in MED-6820) and crosslinker were added and the entire composition (Part A) mixed and degassed under pressure in a vacuum oven for 10 minutes. Finally, to Part A, 35% of base polymer along with the platinum catalyst (Part B) were added and the composition mixed and degassed under pressure. The final formulation was transferred to a syringe. Using 20-gauge needle, it was injected in a bubble of a bubble wrap plastic or in a sac made by gluing the edges of two contact lenses. The silicone was allowed to cure for a period of 24 hours at 35° C. The cured material conformed to the shape of the bubble and possessed desired mechanical properties.

### EXAMPLE V

The porcine or cadaver eye was processed and mounted on a glass slide. The lens was extracted from the capsular bag by performing cataract surgery using an appropriate phacoemulsification machine. The injectable LAL formulation from Examples I-IV was injected into the porcine or cadaver capsular bag following removal of the lens using a syringe equipped with the appropriate gauge of cannula for 1-2 mm incision. The capsular bag containing the LAL was allowed to cure in a water bath at 35° C. overnight. The cured material was adhered to the bag, conformed to the shape of the capsular bag and possessed desired mechanical properties.

### EXAMPLE VI

By mixing 0.83% of initiator (benzoin-polysiloxane-benzoin), 0.04% of UV-absorber (UVAM-polysiloxane-UVAM) in 25% 700 g/mole bismethacrylate endcapped polydimethylsiloxane (macromer), the refraction modulating composition was made. To this mixture, 39% of base polymer (LSR-9, Part A, from Nusil, Inc.) was added and the entire composition (Modified Part A) mixed and degassed under vacuum for 10 minutes. Finally to this Modified Part A, 35% of base polymer along with the platinum catalyst (Part B) was added and the composition mixed and degassed under pressure. The final formulation was stored at ˜−4 to 0° C. in a freezer. The formulation is brought to room temperature and transferred to a syringe and using a 20-gauge needle injected in the capsular bag as described below. The cured material was adhered to the bag, conformed to the shape of the capsular bag and possessed desired mechanical properties.

### EXAMPLE VII

A human cadaver eye was processed and prepared for surgery. The cornea and iris were removed to facilitate the removal of the lens and implantation of the light adjustable lens.

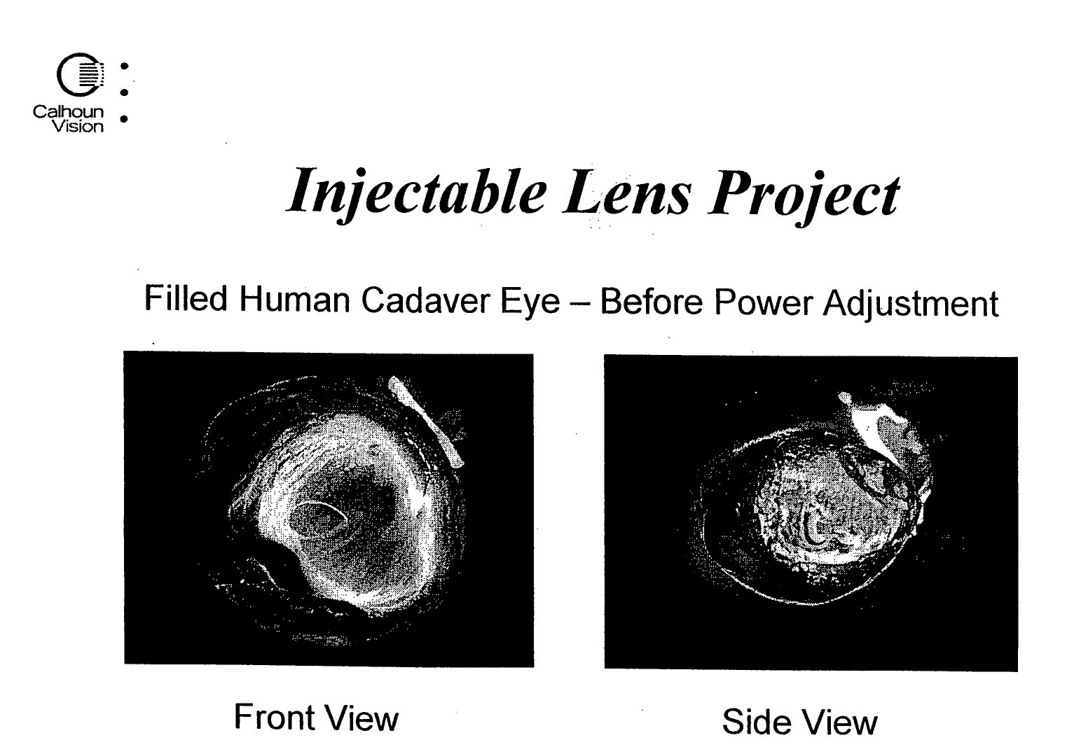

An upper minicircular capsulorhexis of 1.2-1.9 mm was performed on the eye followed by extraction of the lens using a 1 mm phaco tip. Fluid remaining in the capsular bag was removed by pressing on the capsular bag. This was followed by insertion of a silicone plug. A light adjustable formulation similar to that described in Example VI was injected into the capsular bag using a 20-gauge cannula via an EFD dispenser until the capsular bag was filled. The silicone plug was then used to seal the capsular bag. The lens was then cured at 37° C. for over 24 hours. The cured material was adhered to the bag, conformed to the shape of the capsular bag and possessed desired mechanical properties. FIG. 2 shows photographs of the filled capsular bag after curing.

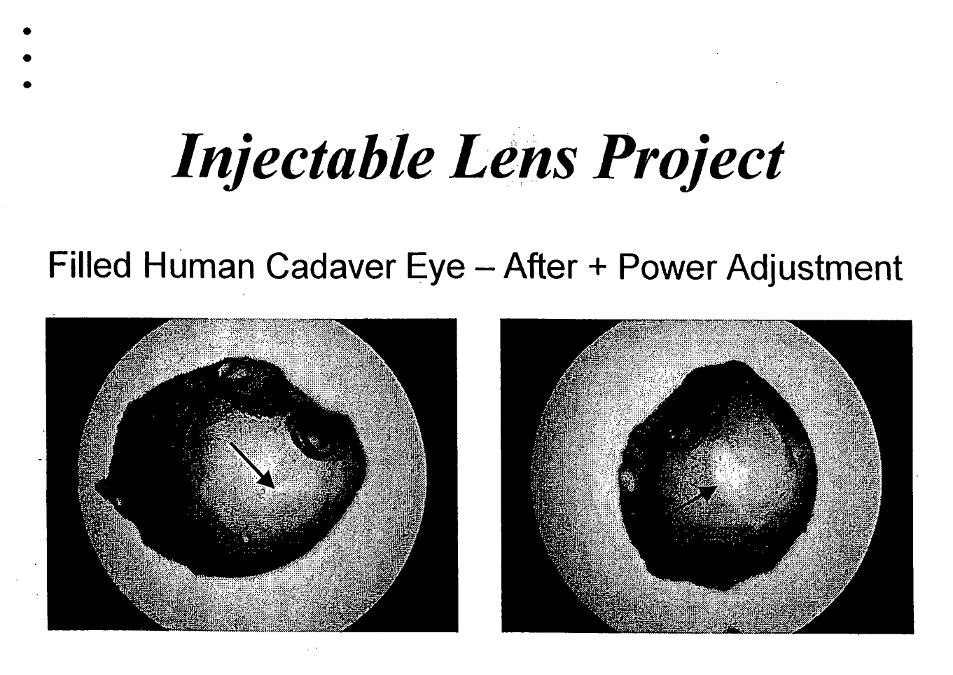

Following curing, a portion of the cured lens with unreacted refraction modulating composition present in it was exposed to UV radiation. This caused localized polymerization of the refraction modulating composition, causing a positive power adjustment in a portion of the lens. FIG. 3 shows photographs of the cured lens after the positive power adjustment.

In each of the examples above, a portion of the lens is exposed to laser energy causing the formation of a second polymer network from the refraction modulating composition comprising of macromer, photoinitiator and UV-absorber dispersed in the first polymer matrix. Formation of the second polymer network causes depletion of the macromer in the irradiated region. This in turn causes migration of macromer from the unexposed regions into the exposed region. This results in a change in the radius of curvature and thus the power of the lens.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for producing a shaped implant comprising:
forming a first polymer matrix in a body cavity, said first polymer matrix having macromers dispersed throughout the first polymer matrix;
exposing at least a portion of said first polymer matrix to an external stimulus such that the macromers form a second polymer matrix.

2. The method of claim 1 wherein said first polymer matrix is formed from monomers selected from the group comprising polyalkyl acrylates, poly-hydroalkyl acrylates, polyvinyls and poly-silicones.

3. The method of claim 1 wherein said first polymer matrix comprises a polysiloxane.

4. The method of claim 3 wherein said polysiloxane is endcapped with a terminal monomer, said monomer selected from the group comprising acetoxy, amino, alkoxy, halide, hydroxy, vinyl, hydride and mercapto monomers.

5. The method of claim 4 wherein said endcapped terminal monomer is bis(diacetoxymethylsilyl)-polydimethylsiloxane.

6. The method of claim 1 wherein the macromers comprises polysiloxanes.

7. The method of claim 6 wherein said polysiloxanes contain a functional group capable of stimulus-induced polymerization.

8. The method of claim 7 wherein said functional group is selected from the group comprising acrylate, allyloxy, cinnamoyl, methacrylate, stibenyl and vinyl.

9. The method of claim 1 wherein the implant further comprises a photoinitiator.

10. The method of claim 9 wherein said photoinitiator is selected from the group comprising: acetophenones, 2,4-dichloromethyl-1,3,5-triazines, benzoin methyl ether, o-benzolyoximinoketone and silicone derivatives thereof.

11. The method of claim 1 wherein said external stimulus is in the form of energy such as heat or light or of electromagnetic origin.

12. The method of claim 1 wherein said implant is an intraoccular lens.

13. The intraoccular lens of claim 12 wherein the lens is an accommodating lens.

14. The intraoccular lens of claim 12 wherein the lens may be corrected for myopia, hyperopia, astigmatism or higher order aberations.

15. A shapable implant comprising:
a first polymer matrix formed in vivo;
macromers dispersed throughout the first polymer matrix; the macromers being capable of forming a second polymer matrix when exposed to an external stimulus.

16. The implant of claim 15 wherein said first polymer matrix is formed from monomers selected from the group comprising polyalkyl acrylates, polyhydroalkyl acrylates, polyvinyls, polyphosphazenes, polyurethanes and polysilicones.

17. The implant of claim 15 wherein said first polymer matrix is prepared from monomers comprising polysiloxanes.

18. The implant of claim 12 wherein said polysiloxane contains an endcapped terminal monomer, said terminal monomer selected from the group comprising acetoxy, amino, alkoxy, halide, hydroxy, vinyl, hydride and mercapto monomers.

19. The implant of claim 15 wherein the macromers comprises polysiloxanes.

20. The implant of claim 19 wherein said polysiloxane contains a functional group capable of stimulus-induced polymerization.

21. The implant of claim 15 further comprising a photoinitiator.

22. The implant of claim 21 wherein said photoinitiator is selected from the group comprising: acetophenones, 2,4-dichloro-methyl-1,3,5-triazines, benzoin methyl ether, o-benzolyoximinoketone and silicone derivatives thereof.

23. The implant of claim 15 further comprising a UV-absorber.

24. The implant of claim 15 wherein said implant is an intraoccular lens.

25. The intraoccular lens of claim 24 wherein said lens is accommodating.

26. The intraoccular lens of claim 24 having a refractive index of from about 1.40 to about 1.50.

27. The implant in claim 15 wherein the stimulus causes a desired change in modulus in the exposed region.

28. A method for preparing a shapable implant comprising:
(a) preparing a first composite, said first composite comprising a first precursor and macromer;
(b) preparing a second composite, said second composite comprising a second precursor and a catalyst capable of inducing the polymerization of said first and second precursors,
(c) combining said first and second composites;
(d) injecting the combined first and second composites into a body cavity;
(e) forming a polymer matrix from said first and second precursors in said body cavity to form an implant, said polymer matrix having the macromer dispersed therein.

29. The method of claim 28 wherein said first precursor comprises a polyorganosiloxane composition having at least two vinyl radicals per molecule.

30. The method of claim 28 wherein said second precursor comprises an organosilicon compound containing at least 2 silicon-bonded hydride groups.

31. The method of claim 28 wherein said catalyst is a platinum group metal catalyst.

32. The method of claim 28 wherein said first composite further comprises a photoinitiator.

33. The method of claim 28 further comprising the step of exposing said implant to an external stimulus to induce polymerization of the macromers.

34. The method of claim 33 wherein said external stimulus is light.

35. The method of claim 28 wherein said body cavity is a capsular bag.

36. An implant formed in vivo wherein the properties of said implant can be modified after formation.

37. The implant of claim 36 further comprising macromers dispersed throughout said implant.

38. The implant of claim 37 wherein said macromers are capable of external stimulus induced polymerization within said implant.

39. The implant of claim 38 wherein said external stimulus is light.

40. The implant of claim 39 wherein said external stimulus is ultraviolet light.

41. The implant of claim 37 wherein said macromers comprise polysiloxanes.

42. The implant of claim 41 wherein said polysiloxanes contain a functional group capable of external stimulus induced polymerization.

43. The implant of claim 42 wherein said functional group is selected from the group comprising acrylate, alloxy, cinnamoyl, methacrylate, stibenyl and vinyl.

44. The implant of claim 36 further comprising a photoinitiator dispersed throughout said implant.

45. The implant of claim 44 wherein said photoinitiator is selected from the group consisting of acetophenones, 2,4-dichloromethyl-1,3,5-triazines, benzoin methyl ether, o-benzol oimino ketone and silicone derivatives thereof.

46. The implant of claim 36 wherein said implant comprises an intraoccular lens.

47. A method for shaping an implant comprising:
(a) forming an implant in vivo; said implant having macromers dispersed therein;
(b) exposing at least a portion of said implant to an external stimulus such that said external stimulus induces a change in the shape of said implant.

48. The method of claim 47 wherein the implant is formed in vivo by
(a) preparing a first composite comprising a first precursor and macromers;
(b) preparing a second composite comprising a second precursor;
(c) combining said first and second composites such that the first and second composites are to form the implant in vivo.

49. The method of claim 48 wherein said second composite further comprises a catalyst.

50. The method of claim 48 wherein said first composite further comprises a photoinitiator.

51. The method of claim 48 further comprising the step of exposing said implant to an external stimulus to induce a change in properties of said implant.

52. The method of claim 47 wherein said change in properties is induced by polymerization of said macromers.

53. The method of claim 52 wherein said external stimulus is light.

54. The method of claim 53 wherein said light is ultraviolet light.

55. The method of claim 48 wherein said first precursor is a polyorganosiloxane composition having an average of at least 2 vinyl radicals per molecule.

56. The method of claim 48 wherein said second precursor comprises an organosilicon compound containing at least 2 silicon-bonded hydride groups.

57. The method of claim 48 wherein the first and second composites are combined in the presence of a platinum group metal catalyst.

58. The method of claim 48 wherein said implant is an intraoccular lens.
